(19) United States
(12) Patent Application Publication
(10) Pub. No.: US 2013/0046288 A1
(43) Pub. Date: Feb. 21, 2013

(54) METHOD AND APPARATUS FOR REMOVING NOXIOUS MATERIALS FROM CELLS

(71) Applicant: Irving N. WEINBERG, Bethesda, MD (US)

(72) Inventor: Irving N. WEINBERG, Bethesda, MD (US)

(73) Assignee: WEINBERG MEDICAL PHYSICS LLC, Bethesda, MD (US)

(21) Appl. No.: 13/213,537

(22) Filed: Aug. 19, 2011

(51) Int. Cl. A61M 1/00

(52) U.S. Cl. 604/540

(57) ABSTRACT

A method and apparatus are provided for removing noxious or unneeded materials from cells in a subject's body.

### FIELD OF THE INVENTION

The presently disclosed embodiments relate to a novel approach for removing noxious material from cells.

### BACKGROUND

Cells in nature behave in a manner to maintain homeostasis, which is the regulation of the cell's internal environment for maintaining a stable, constant condition. However, cells may also create waste products as a result of the different chemical reactions used by the cell to survive. Thos chemical reactions may create waste that requires disposal in order to promote and maintain homeostasis and proper cell operation.

There are natural mechanisms for disposing of cellular waste such as the use of lysosomes and ubiquitin, Additionally, cells may use what have been termed “scavenger molecules” that can bind to toxic molecules and waste substances.

### SUMMARY

However, various medical conditions and/or medical treatment for various medical conditions may create additional amounts of cellular waste that are not readily disposed of by cells using naturally occurring behaviors and methods. Thus, in accordance with various disclosed embodiments, a method and apparatus is provided for removing noxious or unneeded materials from cells in a subject's body. Accordingly, the presently disclosed embodiments may be used in conjunction with medical treatment for various conditions, as explained herein.

### BRIEF DESCRIPTION OF THE FIGURES

A more compete understanding of the present invention and the utility thereof may be acquired by referring to the following description in consideration of the accompanying drawings, in which like reference numbers indicate like features, and wherein:

FIG. 1 illustrates an example of various delivery vehicles containing magnetic particles with binding capabilities that may be brought into a region of cells containing noxious materials in accordance with the disclosed embodiments.

FIG. 2 illustrates a stage of a method for removing noxious materials from a cell, wherein payload of delivery vehicles enters cells containing noxious materials in accordance with the disclosed embodiments.

FIG. 3 illustrates a stage of a method for removing noxious materials from a cell, wherein payload of delivery vehicles binds to noxious materials in accordance with the disclosed embodiments.

FIG. 4 illustrates a stage of a method for removing noxious materials from a cell, wherein payload of delivery vehicles, still bound to noxious materials, exits from the cell, without destroying cell in accordance with the disclosed embodiments.

FIG. 5 illustrates a stage of a method for removing noxious materials from a cell, wherein noxious materials, now removed from cell, are eliminated by nearby glial cells in accordance with the disclosed embodiments.

### DETAILED DESCRIPTION

The description of specific embodiments is not intended to be limiting of the present invention. To the contrary, those skilled in the art should appreciate that there are numerous variations and equivalents that may be employed without departing from the scope of the present invention. Those equivalents and variations are intended to be encompassed by the present invention.

In the following description of various invention embodiments, reference is made to the accompanying drawings, which form a part hereof, and in which is shown, by way of illustration, various embodiments in which the invention may be practiced. It is to be understood that other embodiments may be utilized and structural and functional modifications may be made without departing from the scope and spirit of the present invention.

In accordance with disclosed embodiments, an inventive concept implemented in a method and apparatus(es) remove noxious or unneeded materials from cells in a subject's body. For the purposes of this disclosure, it should be understood that the term “noxious” refers to material which, upon excessive accumulation in a cell, can negatively affect the function of that cell. Hence the noxious material at low concentrations need not be poisonous.

It should be further understood and appreciated that here are various disease states relating to accumulations of such materials, for example the excessive accumulation of mucopolysaccharides in lysosomal storage disease, or of glycogen in Pompe disease. As another example, some or all of the ill effects of Alzheimer disease may be due to accumulation of abnormal proteins (called tau proteins) in neuronal cells. Typical medical interventions which call for removal of diseased cells might work in oncology but are not appropriate in storage diseases, since removal of affected cells would result in abnormal bodily functions.

Further, it should be appreciated that the detrimental side effects of various types of medical treatment may be reduced or eliminated by assisting cells in disposal of noxious material resulting from the administration of the medial treatment on the subject.

Additionally, the efficacy of various types of medical treatments may be improved by implementing the disclosed embodiments in conjunction with those treatments' administration on a subject.

Accordingly, a method and an apparatus(es) for assisting a cell in the removal of noxious material is described in conjunction with the disclosed embodiments are described in conjunction with the figures. The method may include several operational stages.

In a first stage, delivery vehicles are transported to one or more regions of the subject's body affected by accumulation of noxious materials in the affected cells of those regions, where the term “noxious” is defined above. The delivery vehicles may have such properties that they can enter into cells, bind within the cells to the noxious material, and then be removed from the cells with the noxious material attached thereto.

It should be understood that the conformation, i.e., the structure or outline of an item or entity, determined by the arrangement of its parts of the delivery vehicle may change at various stages of the process. In one disclosed embodiment, once the delivery vehicles have been removed from the affected cells (with the noxious material attached or in tow) the noxious materials may be destroyed by other cells (e.g., glial cells in the brain). In such an embodiment, the delivery vehicles may be “magnetic particles,” which term includes the case of magnetic nanoparticles, which may be configured with or without surface or coatings.

In an alternative embodiment, the delivery vehicles may be microbubbles with internal or surface magnetic nanoparticles.

In an alternative embodiment the delivery vehicles may be elongated, as in so-called “magnetic nanoworms.”

In an alternative embodiment the delivery vehicles may themselves be cells or other living organisms.

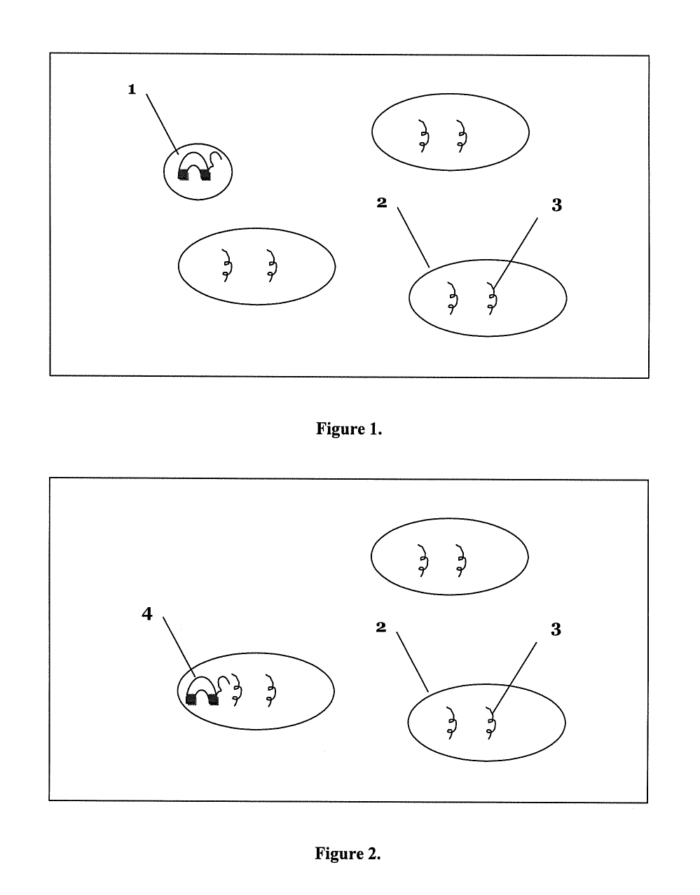

FIG. 1 illustrates an example of various delivery vehicles containing magnetic particles with binding capabilities that may be brought into a region of cells containing noxious materials in accordance with the disclosed embodiments. As shown in FIG. 1, the delivery vehicles contain magnetic particles with binding capabilities 1 that are brought into a region of cells 2 containing noxious materials 3.

Thus, in accordance with disclosed embodiments, the delivery vehicles 1 may be brought to the vicinity of cells 2 containing unwanted or abnormally high concentrations of noxious substances 3. The delivery vehicles may be transported to the region of affected cells via a locally-placed catheter, or via magnetic guidance from the systemic or cerebrospinal circulation or a nearby-placed catheter, or via pores in natural orifices such as the cribiform plate (with or without magnetic guidance). It is known that it is possible to design magnetic nanoparticles to enter cells, as shown in the article: “Magnetic nanoparticles in MR imaging and drug delivery”, by C Sun et al, published in Advanced Drug Delivery Reviews 60:1252-1265 (2008) (the disclosure of which being incorporated by reference in its entirety). It is also known that it is possible to concentrate magnetically-loaded cells into certain regions of the body, as shown in the article: “Magnetically Labeled Neural Progenitor Cells, Which are Localized by Magnetic Force, Promote Axon Growth in Organotypic Cocultures”, by T Hamasaki et al, published in SPINE 32(21):2300-2305 (2007) (the disclosure of which being incorporated by reference in its entirety), and the thesis: “Adaptive Control for the Position of Magnetic Particles Using Magnetic Traps” by J G Pickel, published by the University of Pittsburgh in 2007 (the disclosure of which being incorporated by reference in its entirety).

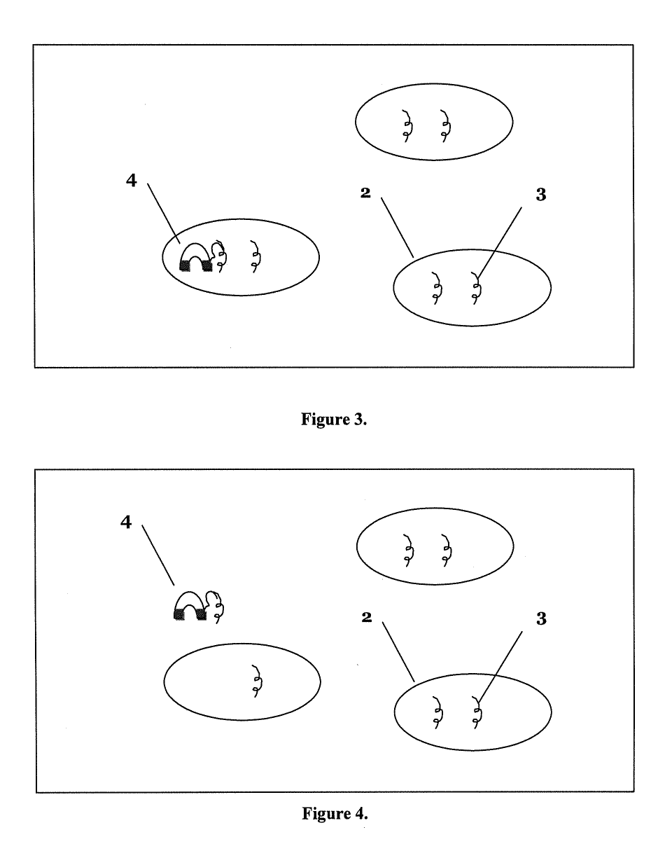

FIG. 2 illustrates a stage of a method for removing noxious materials from a cell, wherein payload of delivery vehicles enters cells containing noxious materials in accordance with the disclosed embodiments. As shown in FIG. 2, these delivery vehicles 4 enter the cells 2 that contain the noxious materials 3.

As shown in FIG. 2, entry of the delivery vehicle 1 into cells 2 may be as a result of a concentration gradient of delivery vehicles between the space outside the affected cells and the interior of the affected cells. Such a gradient could be established through the use of magnetic forces acting upon delivery vehicles containing magnetizable or magnetic particles, in which the magnetic forces created a high extracellular concentration of the particles in the affected region.

In an alternative embodiment, the delivery vehicles may enter the cells by phagocytosis.

In yet another alternative embodiment, the delivery vehicles may enter the cells via active transport.

In an alternative embodiment, the delivery vehicles may enter the cell or cell part and release their payload (e.g., of magnetic particles) 4.

In another alternative embodiment, electromagnetic radiation (for example, radiofrequency) can be applied to disrupt the natural barriers to transport, via electroporation, local heating or other mechanisms, for example as shown in the article “Radiofrequency Studies on Tumorigenesis and Blood-Brain Barrier in Lab Animals Support the Conclusion of No Adverse Effects without Significant Tissue Temperature Increase” by J A Elder, published in the Proceedings of the 2010 Asia-Pacific International Symposium on Electromagnetic Compatibility (the disclosure of which being incorporated by reference in its entirety).

It should be appreciated that mechanisms may be used to transport and/or manipulate the delivery vehicle during various stages of operation including delivery of the delivery vehicle to and/or from a region of a subject's body, to facilitate or trigger entry of the delivery vehicle into the cell or cell part, to facilitate or trigger binding of the payload with the noxious materials, and/or to facilitate exit of the bound noxious materials from the cell or cell part.

Once within the cells, the payload of the delivery vehicle may bind with the noxious material to be removed. FIG. 3 illustrates a stage of a method for removing noxious materials from a cell, wherein payload of delivery vehicles binds to noxious materials in accordance with the disclosed embodiments. As shown in FIG. 3, the payload of the delivery vehicles 4 binds to the noxious materials 3.

As illustrated in FIG. 3, the delivery vehicles (or, alternatively only their payloads) bind to the noxious materials in the cell. As an example, it is known that cells can tag abnormal proteins with ubiquitin, and that it is possible to make ligands that bind to the ubiquitin. It is known that it is possible to build magnetic nanoparticles that selectively bind certain proteins, as shown in the article “Towards oriented assembly of proteins into magnetic nanoparticles”, by C-W Hung, T R P Holoman, P Kofinas, W E Bently, published in the Biochemical Engineering Journal 38:164-170 (2008) (the disclosure of which being incorporated by reference in its entirety). It is also possible to make ligands that bind to tau protein in cells from the brains of patients with Alzheimer disease, as is shown in the article “Interaction of tau protein with the dynactin complex”, by E Magnani et al, published by the EMBO Journal 26:4546-4554 (2007) (the disclosure of which being incorporated by reference in its entirety).

It is also known that it is possible to bind ubiquitin to magnetic nanoparticles, as shown in “A putative, ubiquitin-dependent mechanism for the recognition and elimination of defective spermatozoa in the mammalian epididymis”, by P Sutovsky et al, published in the Journal of Cell Science 114:1665-1675 (2001) (the disclosure of which being incorporated by reference in its entirety). The article by P Sutovsky cited above also demonstrates how the body may eliminate ubiquitin-tagged cells naturally.

In an alternative embodiment, the conformation of the delivery vehicles or their payloads is altered, for example by application of electromagnetic radiation or electric or magnetic fields. In an alternative embodiment, the presence of the magnetic nanoparticles, whether or not under the influence of an external magnetic field, may alter the conformation of the noxious material in order to render it less noxious, with or without the need to remove the noxious material from the cell.

FIG. 4 illustrates a stage of a method for removing noxious materials from a cell, wherein payload of delivery vehicles, still bound to noxious materials, exits from the cell, without destroying cell in accordance with the disclosed embodiments. As shown in FIG. 4, the payload of the delivery vehicles 4 remains bound to the noxious material during the exit of the payload material from the cell. Of particular note, this exit of the material does not destroy the cell.

The delivery vehicles (or, alternatively, only their payloads), now bound to the noxious materials, may be removed from the affected cells, or from internal parts of the affected cells (e.g., the nucleus) using a number of techniques. Fore example, in accordance with at least one disclosed embodiment, the method of removal may include applying magnetic forces or gradients that act on magnetic particles to physically establish a velocity vector radiating outward from the cell or internal cell part.

Alternatively, or in addition, the concentration gradient of the magnetic particles or delivery vehicles may be such that there is a tendency for them to exit from the cell or from the internal cell part without external manipulation. However, it should also be understood that the establishment of magnetic gradients may assist in creating such a concentration gradient.

In another alternative embodiment, the removal of the delivery vehicles may be enabled or promoted via exocytosis or other means of disposal from the cell or cell part.

In another alternative embodiment, electromagnetic radiation (for example, radiofrequency) can be applied to disrupt the natural barriers to transport, via electroporation or other mechanisms.

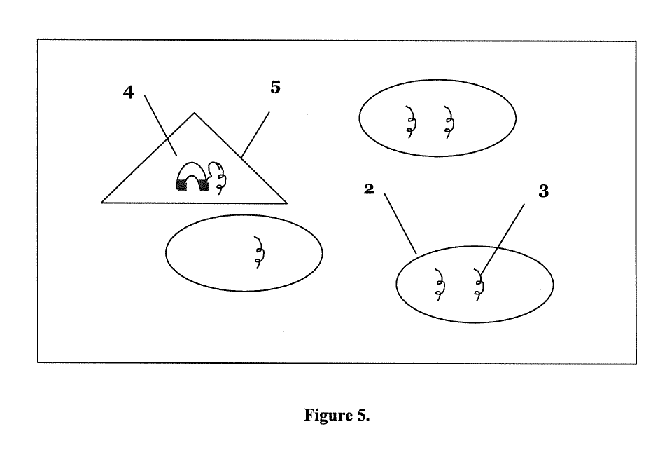

FIG. 5 illustrates a stage of a method for removing noxious materials from a cell, wherein noxious materials, now removed from cell, are eliminated by nearby glial cells in accordance with the disclosed embodiments. Thus, a shown in FIG. 5, the noxious materials 3 may be removed from cell and subsequently eliminated by nearby glial cell 5 or some other naturally occurring or artificially induced mechanism.

The removed noxious materials, whether bound or unbound to the delivery vehicles (or the delivery vehicles' payloads) may be destroyed, neutralized or removed via various natural or artificially induced mechanisms. For example, this destruction may be implemented via natural defenders of the subject's own body, such as glial cells 5, that can act in the extracellular space but may be unable to act effectively on noxious materials that are inside other cells.

It is known, for example, that microglia can remove magnetic nanoparticles, as shown in the article “Robust Uptake of Magnetic Nanoparticles (MNPs) by Central Nervous System (CNS) Microglia: Implications for Particle Uptake in Mixed Neural Cell Populations”, by MR Pickard and DM Chari, published in Int. J. Mol. Sci. 11:967-981 (2010) (the disclosure of which being incorporated by reference in its entirety).

In an alternative embodiment, the noxious materials are removed via diffusion or other natural transport processes and eliminated from the body. The noxious materials may be transported in the bound state to the delivery vehicles (or the delivery vehicles' payloads) or dissociated from the bound state.

While the disclosed embodiments of this inventive concept have been described in conjunction with the specific embodiments outlined above, it is evident that many alternatives, modifications and variations will be apparent to those skilled in the art. Accordingly, the various embodiments of the invention, as set forth above, are intended to be illustrative, not limiting. Various changes may be made without departing from the spirit and scope of the invention.

Additionally, it should be understood that the functionality described in connection with various described components of various invention embodiments may be combined or separated from one another in such a way that the architecture of the invention is somewhat different than what is expressly disclosed herein. Moreover, it should be understood that, unless otherwise specified, there is no essential requirement that methodology operations be performed in the illustrated order; therefore, one of ordinary skill in the art would recognize that some operations may be performed in one or more alternative order and/or simultaneously.

Various components of the invention may be provided in alternative combinations operated by, under the control of or on the behalf of various different entities or individuals.

As a result, it will be apparent for those skilled in the art that the illustrative embodiments described are only examples and that various modifications can be made within the scope of the invention as defined in the appended claims.

What is claimed:

1. A system for removing noxious materials from cells or parts of cells of a subject's body, comprising:
a delivery vehicle configured to enter into and exit from cells and/or parts of cells;
a payload provided within the delivery vehicle, the payload including magnetic particles; and
an apparatus configured to concentrate and/or manipulate the delivery vehicle
wherein the delivery vehicle or its payload bind to the noxious materials.

2. The system of claim 1, wherein the system is used to remove excessive accumulation of mucopolysaccharides in lysosomal storage disease.

3. The system of claim 1, wherein the system is used to remove excessive accumulation of glycogen in Pompe disease.

4. The system of claim 1, wherein the system is used to reduce the effects of Alzheimer disease by reducing accumulation of abnormal proteins) in neuronal cells.

5. The system of claim 1, wherein the system is used to reduce side effects of a medical treatment by assisting cells in disposal of noxious material resulting from the administration of the medial treatment on the subject.

6. The system of claim 1, wherein the system is used to improve the efficacy of a medical treatment on a subject.

7. The system of claim 1, wherein noxious material is material which, upon excessive accumulation in a cell, can negatively affect the function of that cell.

8. The system of claim 1, wherein the conformation of the delivery vehicle changes prior to the delivery of the noxious material to a position outside the cell.

9. The system of claim 1, wherein the conformation of the delivery vehicle changes as a result of operation of the apparatus.

10. The system of claim 1, wherein the delivery vehicle includes a magnetic particle, a magnetic nanoparticle, a microbubble with internal or surface magnetic nanoparticles, a magnetic nanoworm, or a cell or other living organism.

11. The system of claim 1, wherein the delivery vehicle is transported to the region of affected cells via a locally-placed catheter, via magnetic guidance from systemic or cerebrospinal circulation or a nearby-placed catheter, or via pores in natural orifices such as the cribiform plate with or without magnetic guidance.

12. The system of claim 1, wherein the delivery vehicle enters the affected cell or cell part as a result of a concentration gradient of delivery vehicles between a space outside the affected cell and the interior of the affected cell.

13. The system of clam 12, wherein the concentration gradient is established through the use of magnetic forces acting upon the delivery vehicle.

14. The system of claim 1, wherein the delivery vehicle enters the affected cell or cell part as a result of phagocytosis.

15. The system of claim 1, wherein the delivery vehicle enters the affected cell of cell part as a result of active transport.

16. The system of claim 1, wherein, following the delivery vehicle entering the cell or cell part, the delivery vehicle releases its payload.

17. The system of claim 1, wherein the conformation of the delivery vehicle or the payload is altered by application of electromagnetic radiation in order to bind the noxious material.

18. The system of claim 1, wherein the bound noxious material is removed from the cell by application of magnetic forces or gradients that physically establish a velocity vector radiating outward from the cell or cell part.

19. The system of claim 1, wherein the bound noxious material is removed from the cell via exocytosis.

20. A method for removing noxious materials from cells or parts of cells, comprising:
transporting a delivery vehicle to a region of a subject's body, the delivery vehicle being configured to enter into and exit from cells and/or parts of cells; the delivery vehicle including a payload including magnetic particles; and
triggering entry of the delivery vehicle including the payload into the cells and/or cell parts containing noxious materials;
triggering binding of the payload of the delivery vehicle to the noxious material; and
triggering exit of the payload bound with the noxious material from the cell and/or cell part.

21. The method of claim 20, wherein the removal of the noxious material is used to remove excessive accumulation of mucopolysaccharides in lysosomal storage disease.

22. The method of claim 20, wherein the removal of the noxious material is used to remove excessive accumulation of glycogen in Pompe disease.

23. The method of claim 20, wherein the removal of the noxious material is used to reduce the effects of Alzheimer disease by reducing accumulation of abnormal proteins) in neuronal cells.

24. The method of claim 20, wherein the removal of the noxious material is used to reduce side effects of a medical treatment by assisting cells in disposal of noxious material resulting from the administration of the medial treatment on the subject.

25. The method of claim 20, wherein the removal of the noxious material is used to improve the efficacy of a medical treatment on a subject.

26. The method of claim 20, wherein noxious material is material which, upon excessive accumulation in a cell, can negatively affect the function of that cell.

27. The method of claim 20, wherein triggering of entry of the delivery vehicle, triggering binding and/or triggering exist of the payload is caused by inducing conformation of the delivery vehicle and/or payload via application of electromagnetic radiation.

28. The method of claim 20, wherein the delivery vehicle includes a magnetic particle, a magnetic nanoparticle, a microbubble with internal or surface magnetic nanoparticles, a magnetic nanoworm, or a cell or other living organism.

29. The method of claim 20, wherein the delivery vehicle is transported to the region of affected cells via a locally-placed catheter, via magnetic guidance from systemic or cerebrospinal circulation or a nearby-placed catheter, or via pores in natural orifices such as the cribiform plate with or without magnetic guidance.

30. The method of claim 20, wherein the delivery vehicle enters the affected cell or cell part as a result of a concentration gradient of delivery vehicles between a space outside the affected cell and the interior of the affected cell.

31. The method of clam 30, wherein the concentration gradient is established through the use of magnetic forces acting upon the delivery vehicle.

32. The method of claim 20, wherein the delivery vehicle enters the affected cell or cell part as a result of phagocytosis.

33. The method of claim 20, wherein the delivery vehicle enters the affected cell of cell part as a result of active transport.

34. The method of claim 20, wherein, following the delivery vehicle entering the cell or cell part, the delivery vehicle releases its payload.

35. The method of claim 20, wherein the bound noxious material is removed from the cell by application of magnetic forces or gradients that physically establish a velocity vector radiating outward from the cell or cell part.

36. The method of claim 20, wherein the bound noxious material is removed from the cell via exocytosis.

37. The method of claim 20, wherein the bound noxious material is eliminated from the subject's body via a naturally occurring mechanism of the subject's body.

38. The method of claim 20, wherein the bound noxious material is eliminated from the subject's body via an artificially induced mechanism.
